(19) United States
(12) Patent Application Publication
(10) Pub. No.: US 2010/0173941 A1
(43) Pub. Date: Jul. 8, 2010

(54) Muscarinic Receptor Agonists that are Effective in the Treatment of Pain, Alzheimer's Disease and Schizophrenia

(75) Inventors: Yun-Xing Cheng, Saint Laurent (CA); Miroslaw Tomaszewski, Saint Laurent (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/303,637

(22) PCT Filed: Jun. 8, 2007
(86) PCT No.: PCT/SE07/00555

§ 371 Date: Dec. 5, 2008

Related U.S. Application Data
(60) Provisional application No. 60/812,416, filed on Jun. 9, 2006.

(51) Int. Cl. A61K 31/454; C07D 405/14; C07D 403/04; A61P 25/28; A61P 29/00; A61P 25/18

(52) U.S. Cl. 514/322; 546/199

(57) ABSTRACT

Compounds of Formulae I, or pharmaceutically acceptable salts thereof: wherein R¹, R², R³, R⁴, R⁵, R⁶, R⁷, m and n are as defined in the specification as well as salts and pharmaceutical compositions including the compounds are prepared. They are useful in therapy, in particular in the management of pain.

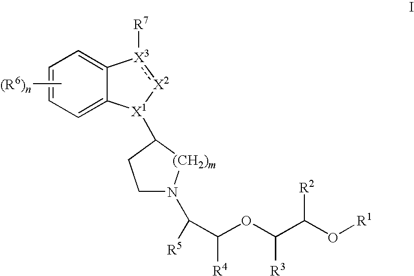

### BACKGROUND OF THE INVENTION

1. Field of the Invention

The present invention relates to agonists of muscarinic receptors. The present invention also provides compositions comprising such agonists, and methods therewith for treating muscarinic receptor mediated diseases. Particularly, the present invention is related to compounds that may be effective in treating pain, Alzheimer's disease, and/or schizophrenia.

2. Discussion of Relevant Technology

The neurotransmitter acetylcholine binds to two types of cholinergic receptors: the ionotropic family of nicotinic receptors and the metabotropic family of muscarinic receptors. Muscarinic receptors belong to the large superfamily of plasma membrane-bound G protein coupled receptors (GPCRs) and show a remarkably high degree of homology across species and receptor subtype. These M1-M5 muscarinic receptors are predominantly expressed within the parasympathetic nervous system which exerts excitatory and inhibitory control over the central and peripheral tissues and participate in a number of physiologic functions, including heart rate, arousal, cognition, sensory processing, and motor control.

Muscarinic agonists such as muscarine and pilocarpine, and antagonists, such as atropine have been known for over a century, but little progress has been made in the discovery of receptor subtype-selective compounds, thereby making it difficult to assign specific functions to the individual receptors. See, e.g., DeLapp, N. et al., “Therapeutic Opportunities for Muscarinic Receptors in the Central Nervous System,” J. Med. Chem., 43(23), pp. 4333-4353 (2000); Hulme, E. C. et al., “Muscarinic Receptor Subtypes,” Ann. Rev. Pharmacol. Toxicol., 30, pp. 633-673 (1990); Caulfield, M. P. et al., “Muscarinic Receptors-Characterization, Coupling, and Function,” Pharmacol. Ther., 58, pp. 319-379 (1993); Caulfield, M. P. et al., International Union of Pharmacology. XVII. Classification of Muscarinic Acetylcholine Receptors,” Pharmacol. Rev., 50, pp. 279-290 (1998).

The Muscarinic family of receptors is the target of a large number of pharmacological agents used for various diseases, including leading drugs for COPD, asthma, urinary incontinence, glaucoma, schizophrenia, Alzheimer's (AchE inhibitors), and Pain.

For example, direct acting muscarinic receptor agonists have been shown to be antinociceptive in a variety of animal models of acute pain (Bartolini A., Ghelardini C., Fantetti L., Malcangio M., Malmberg-Aiello P., Giotti A. Role of muscarinic receptor subtypes in central antinociception. Br. J. Pharmacol. 105:77-82, 1992; Capone F., Aloisi A. M., Carli G., Sacerdote P., Pavone F. Oxotremorine-induced modifications of the behavioral and neuroendocrine responses to formalin pain in male rats. Brain Res. 830:292-300, 1999.).

A few studies have examined the role of muscarinic receptor activation in chronic or neuropathic pain states. In these studies, the direct and indirect elevation of cholinergic tone was shown to ameliorate tactile allodynia after intrathecal administration in a spinal ligation model of neuropathic pain in rats and these effects again were reversed by muscarinic antagonists (Hwang J.-H., Hwang K.-S., Leem J.-K., Park P.-H., Han S.-M., Lee D.-M. The antiallodynic effects of intrathecal cholinesterase inhibitors in a rat model of neuropathic pain. Anesthesiology 90:492-494, 1999; Lee E. J., Sim J. Y, Park J. Y., Hwang J. H., Park P. H., Han S. M. intrathecal carbachol and clonidine produce a synergistic antiallodynic effect in rats with a nerve ligation injury. Can J Anaesth 49:178-84, 2002.). Thus, direct or indirect activation of muscarinic receptors has been shown to elicit both acute analgesic activity and to ameliorate neuropathic pain. Muscarinic agonists and ACHE-Is are not widely used clinically owing to their propensity to induced a plethora of adverse events when administered to humans. The undesirable side-effects include excessive salivation and sweating, enhanced gastrointestinal motility, and bradycardia among other adverse events. These side-effects are associated with the ubiquitous expression of the muscarinic family of receptors throughout the body.

### DESCRIPTION OF THE EMBODIMENTS

To date, five subtypes of muscarinic receptors (M1-M5) have been cloned and sequenced from a variety of species, with differential distributions in the body.

Therefore, it was desirable to provide molecules would permit selective modulation, for example, of muscarinic receptors controlling central nervous function without also activating muscarinic receptors controlling cardiac, gastrointestinal or glandular functions.

There is also a need for methods for treating muscarinic receptor-mediated diseases.

There is also a need for modulators of muscarinic receptors that are selective as to subtypes M1-M5.

The term “Cₘ₋ₙ” or “Cₘ₋ₙ group” refers to any group having m to n carbon atoms.

The term “alkyl” refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkenyl” refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “cycloalkyl” refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” refers to a monovalent ring-containing hydrocarbon radical having at least one carbon-carbon double bond and comprising at least 3 up to about 12 carbon atoms.

The term “aryl” refers to a monovalent hydrocarbon radical having one or more polyunsaturated carbon rings having aromatic character, (e.g., 4n+2 delocalized electrons) and comprising 5 up to about 14 carbon atoms.

The term “heterocycle” refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” refers to a radical derived from a heterocycle by removing one or more hydrogens therefrom.

The term “heterocyclyl” refers a monovalent radical derived from a heterocycle by removing one hydrogen therefrom.

The term “heterocyclylene” refers to a divalent radical derived from a heterocycle by removing two hydrogens therefrom, which serves to links two structures together.

The term “heteroaryl” refers to a heterocyclyl having aromatic character.

The term “heterocylcoalkyl” refers to a monocydic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “heteroarylene” refers to a heterocyclylene having aromatic character.

The term “heterocycloalkylene” refers to a heterocyclylene that does not have aromatic character.

The term “six-membered” refers to a group having a ring that contains six ring atoms.

The term “five-membered” refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, ±1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinudidine diazabicyclo[2.2.1]heptane and 7-oxabicyclo[2.2.1]heptane.

Heterocyclyl includes, for example, monocydic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazotyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

Halogen includes fluorine, chlorine, bromine and iodine.

“RT” or “rt” means room temperature.

In one aspect, an embodiment of the invention provides a compound of Formula I, a pharmaceutically acceptable salt thereof, diastereomers, enantiomers, or mixtures thereof:

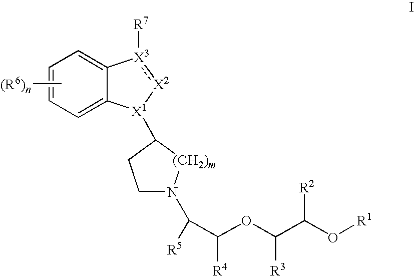

wherein

R¹ is selected from hydrogen, C₁₋₆alkyl, C₂₋₆alkenyl, C₁₋₆alkyl-carbonyl, C₁₋₄alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₆heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂), —OR, R, —C(═O)—R, —CO₂R, —SO₂R, —SO₂NR₂, halogen, —NO₂, —NR₂, —(CH₂)ₚNR₂, and —C(═O)—NR₂; R², R³ and R⁴ are independently selected from hydrogen, C₁₋₆alkyl, C₂₋₆alkenyl, and C₁₋₆alkoxy, wherein said C₁₋₆alkyl, C₂₋₆alkenyl, and C₁₋₆alkoxy are optionally substituted by one or more groups selected from hydroxy, amino, halogen, C₁₋₆alkoxy and —CN; or R³ and R⁴ together with the atoms connected thereto form a 5 to 7 membered C₂₋₆heterocycloalkyl or C₂₋₆heteroaryl; or R¹ and R² together with the atoms connected thereto form a 5 to 7 membered C₂₋₆heterocycloalkyl or C₂₋₆heteroaryl, wherein said C₂₋₆heterocycloalkyl or C₂₋₆heteroaryl is optionally substituted with one or more group selected from C₆₋₁₀aryl, C₂₋₉heteroaryl, C₃₋₆heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₚOR, R, —CO₂R; —SO₂R; —SO₂NR₂, halogen, —NO₂, —NR₂, —(CH₂)ₚNR₂, and —C(═O)—NR₂;

R⁵ is selected from hydrogen, halogen, hydroxy, C₁₋₆alkyl, C₂₋₆alkenyl, and C₁₋₆alkoxy, wherein said C₁₋₆alkyl, C₂₋₆alkenyl, and C₁₋₆alkoxy are optionally substituted by one or more groups selected from hydroxy, amino, halogen, C₁₋₆alkoxy and —CN;

R⁶ is independently selected from hydrogen, halogen, C₁₋₆alkyl, C₂₋₆alkenyl, —CN, —C(═O)—OR, —C(═O)—NR₂, hydroxy, and C₁₋₆alkoxy;

R⁷ is selected from hydrogen, C₁₋₆alkyl, C₂₋₆alkenyl, halogen and C₁₋₆alkoxy;

n is 1, 2, 3 or 4;

m is 1, 2 or 3;

p is 1, 2, 3 or 4;

each R is independently hydrogen, C₁₋₆alkyl, C₂₋₆alkenyl or halogenated C₁₋₆alkyl; and

X¹, X² and X³ is independently selected from C(═O), NH, N, CH₂, and CH, wherein at least one of X¹, X² and X³ is selected from NH and N; wherein at most one of X¹, X² and X³ is C(═O); and wherein X¹ is not C(═O).

In a particular embodiment, R¹ and R² of formula I together with the atoms connected thereto form a 5 to 7 membered C₂₋₆heterocycloalkyl, wherein said C₂₋₆heterocycloalkyl is optionally substituted with one or more groups selected from C₁₋₆alkyl, C₁₋₆alkoxy, hydroxy, halogen, amine and amido.

In another particular embodiment, R³ and R⁴ of formula I together with the atoms connected thereto form a 5 to 7 membered C₂₋₆heteroaryl, wherein said C₂₋₆heteroaryl is optionally substituted with one or more groups selected from C₁₋₆alkyl, C₁₋₆alkoxy, hydroxy, halogen, amine and amido.

In a further particular embodiment, R¹, R², R³ and R⁴ are independently selected from hydrogen, C₁₋₆alkyl, C₂₋₆alkenyl, C₁₋₆alkyl-carbonyl, C₁₋₆alkylaminocarbonyl, and C₁₋₆alkoxy, wherein said C₁₋₆alkyl, C₂₋₆alkenyl, C₁₋₆alkylcarbonyl, C₁₋₆alkylaminocarbonyl, and C₁₋₆alkoxy are optionally substituted by one or more groups selected from amino, halogen, hydroxy, C₁₋₆alkoxy and —CN.

In an even further embodiment, R¹, R², R³ and R⁴ are independently selected from hydrogen, and C₁₋₃alkyl.

In another embodiment, R⁵ is selected from hydrogen and C₁₋₆alkyl optionally substituted by one or more groups selected from hydroxy, amino, halogen, C₁₋₆alkoxy and —CN.

In a further embodiment, R⁵ is hydrogen.

In another embodiment, R⁶ is selected from hydrogen, halogen, methyl, ethyl, —CN, —C(═O)—NH₂, —CO₂CH₃, —CO₂H, hydroxy and methoxy.

In another embodiment, n is 1.

In another embodiment, m is 2.

In a further embodiment, m is 1.

In a further embodiment, p is 1.

In another embodiment, p is 2.

In another embodiment, X¹ is selected from N and CH.

In a further embodiment, X² is selected from N and C(═O).

In an even further embodiment, X³ is selected from N and CH.

In another embodiment, R⁷ is selected from hydrogen, C₁₋₆alkyl and halogen.

In a further embodiment, R⁷ is hydrogen.

In another embodiment, the invention provides a compound of formula II, a pharmaceutically acceptable salt thereof, diastereomer, enantiomer, or mixture thereof:

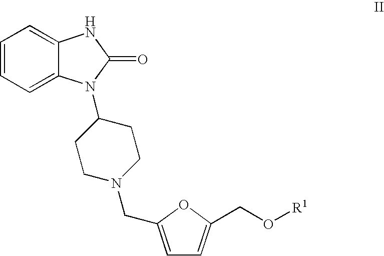

wherein

R¹ is selected from hydrogen, C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₆heterocycloalkyl-C₁₋₃alkyl, C₃₋₆cycloalkyl, and C₃₋₆cycloalkyl-C₁₋₄alkyl, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀-aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂, wherein p is 1, 2, 3 or 4; and each R is independently hydrogen, C₁₋₆alkyl, C₂₋₆alkenyl or halogenated C₁₋₆alkyl.

In a particular embodiment, R¹ of formula II is selected from hydrogen, C₁₋₆alkyl-carbonyl, C₁₋₆alkylaminocarbonyl, C₁₋₆alkyl and C₂₋₆alkenyl, wherein said C₁₋₆alkyl-carbonyl, C₁₋₆alkylaminocarbonyl, C₁₋₆alkyl and C₂₋₆alkenyl are optionally substituted by one or more groups selected from fluoro, chloro, bromo, methoxy, hydroxy and —CN.

In another particular embodiment, R¹ of formula II is selected from hydrogen and C₁₋₃alkyl.

In another embodiment, the invention provides a compound of formula III, a pharmaceutically acceptable salt thereof, diastereomer, enantiomer, or mixture thereof:

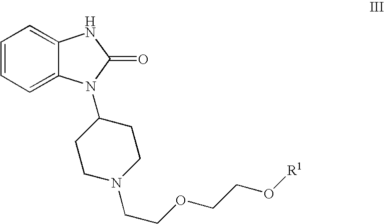

wherein

R¹ is selected from hydrogen, C₁₋₆alkyl, C₂₋₆alkenyl, C₆₋₁₀aryl, C₂₋₆heteroaryl, C₃₋₅heterocycloalkyl, C₆₋₁₀aryl-C₁₋₃alkyl, C₂₋₆heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₆₋₁₀aryl, C₂₋₆heteroaryl, C₃₋₆heterocycloalkyl, C₆₋₁₀aryl-C₁₋₃alkyl, C₂₋₄heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂, wherein p is 1, 2, 3 or 4; and each R is independently hydrogen, C₁₋₆alkyl, C₂₋₃alkenyl or halogenated C₁₋₆alkyl.

In a particular embodiment, R¹ of formula III is selected from hydrogen, C₁₋₆alkyl and C₂₋₆alkenyl, wherein said C₁₋₆alkyl and C₂₋₃alkenyl are optionally substituted by one or more groups selected from fluoro, chloro, bromo, methoxy, hydroxy and —CN.

In another particular embodiment, R¹ of formula III is selected from hydrogen and C₁₋₃alkyl.

In another embodiment, the invention provides a compound of formula IV, a pharmaceutically acceptable salt thereof, diastereomer, enantiomer, or mixture thereof:

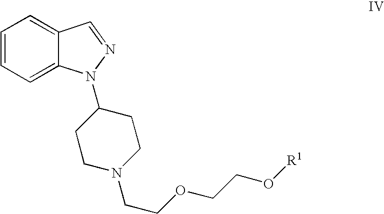

wherein

R¹ is selected from hydrogen, C₁₋₆alkyl, C₂₋₆alkenyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)₉NR₂, and —C(═O)—NR₂, wherein p is 1, 2, 3 or 4; and each R is independently hydrogen, C₁₋₆ alkyl, C₂₋₆alkenyl or halogenated C₁₋₆alkyl.

In a particular embodiment, R¹ of formula IV is selected from hydrogen, C₁₋₆alkyl and C₂₋₆alkenyl, wherein said C₁₋₆alkyl and C₂₋₆alkenyl are optionally substituted by one or more groups selected from fluoro, chloro, bromo, methoxy, hydroxy and —CN.

In another particular embodiment, R¹ of formula IV is selected from hydrogen and C₁₋₃alkyl.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I, II, III or IV. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I, II, III or IV. It will further be understood that the present invention encompasses tautomers of the compounds of the Formula I, II, III or IV.

It will also be understood that certain compounds of the present invention may exist in solvated, for example hydrated, as well as unsolvated forms. It will further be understood that the present invention encompasses all such solvated forms of the compounds of the Formula I, II, III or IV.

Within the scope of the invention are also salts of the compounds of the Formula I, II, III or IV. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I, II, III or IV above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as agonists of M1 receptors. More particularly, the compounds of the invention exhibit selective activity as agonist of the M1 receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of M1 receptors is present or implicated. Furthermore, the compounds of the invention may be used to treat cancer, multiple sclerosis, Parkinson's disease, Huntington's chorea, schizophrenia, Alzheimer's disease, anxiety disorders, obesity, gastrointestinal disorders and cardiovascular disorders.

In a particular embodiment, the compounds may be used to treat schizophrenia or Alzheimer's disease.

In another embodiment, the compounds may be used to treat pain.

In another particular embodiment, the compounds may be used to treat neuropathic pain.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of M1 receptors is present or implicated in that paradigm. This may involve the use of isotopically labeled versions of the compounds of the invention in diagnostic techniques and imaging applications such as positron emission tomography (PET).

Compounds of the invention are useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, obesity, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the Formula I, II, III or IV above, for the manufacture of a medicament for the treatment of any of the conditions discussed above.

A further aspect of the invention is a method for the treatment of a subject suffering from any of the conditions discussed above, whereby an effective amount of a compound according to the Formula I, II, III or IV above, is administered to a patient in need of such treatment.

Thus, the invention provides a compound of Formula I, II, III or IV or pharmaceutically acceptable salt or solvate thereof, as hereinbefore defined for use in therapy.

In a further aspect, the present invention provides the use of a compound of Formula I, II, III or IV or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain. In a particular embodiment, the compounds are useful in therapy for neuropathic pain. In an even more particular embodiment, the compounds are useful in therapy for chronic neuropathic pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, transdermally; intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of Formula I, II, III or IV as defined above for the manufacture of a medicament.

Also within the scope of the invention is the use of any compound of Formula I, II, III or IV for the manufacture of a medicament for the therapy of pain.

Additionally provided is the use of any compound according to Formula I, II, III or IV for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I, II, III or IV above, is administered to a patient in need of such therapy.

Additionally, there is provided a pharmaceutical composition comprising a compound of Formula I, II, III or IV or a pharmaceutically acceptable salt thereof, in association with a pharmaceutically acceptable carrier.

Particularly, there is provided a pharmaceutical composition comprising a compound of Formula I, II, III or IV or a pharmaceutically acceptable salt thereof, in association with a pharmaceutically acceptable carrier for therapy, more particularly for therapy of pain.

Further, there is provided a pharmaceutical composition comprising a compound of Formula I, II, III or IV or a pharmaceutically acceptable salt thereof, in association with a pharmaceutically acceptable carrier use in any of the conditions discussed above.

In a further aspect, the present invention provides a method of preparing the compounds of the present invention.

In one embodiment, the invention provides a process for preparing a compound of Formula I, comprising:

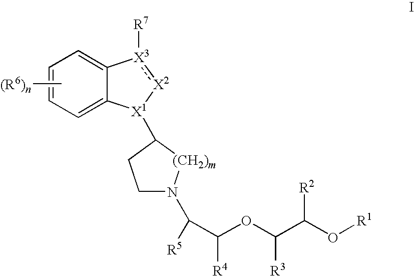

reacting a compound of Formula V with a compound of formula VI,

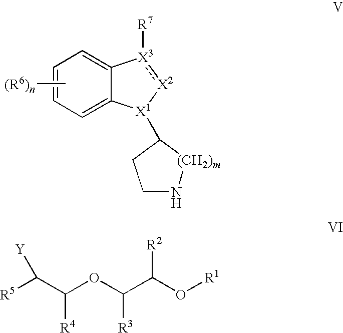

wherein R¹, R², R³, R⁴, R⁵, R⁶, R⁷, m and n are defined above, Y is a halogen.

Optionally, the step of reacting a compound of formula V with a compound of formula VI is carried out in the presence of a base, such as sodium carbonate.

In another embodiment, the invention provides a process for preparing a compound of Formula II, comprising:

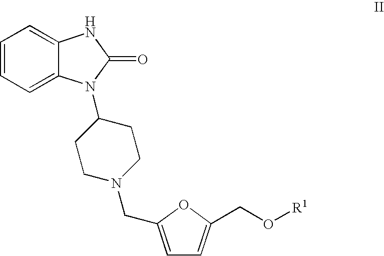

reacting 1-piperidin-4-yl-1,3-dihydro-2H-benzimidazol-2-one with a compound of formula VII,

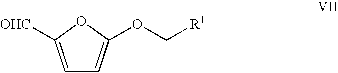

wherein R¹ are defined as above.

Optionally, the step of reacting 1-piperidin-4-yl-1,3-dihydro-2H-benzimidazol-2-one with a compound of formula VII is carried out in the presence of a reducing agent, such as NaBH(OAc)₃ or NaBH₄.

Compounds of the present invention may also be prepared according to the synthetic routes as depicted in Schemes 1-3.

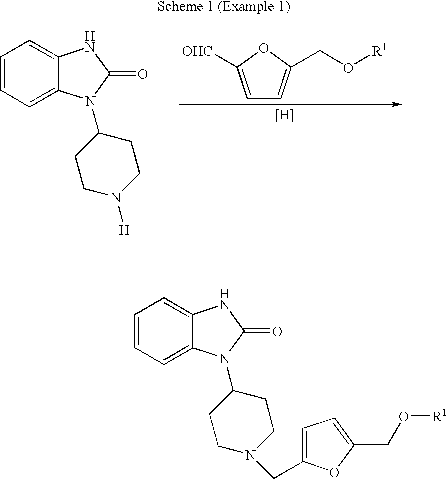

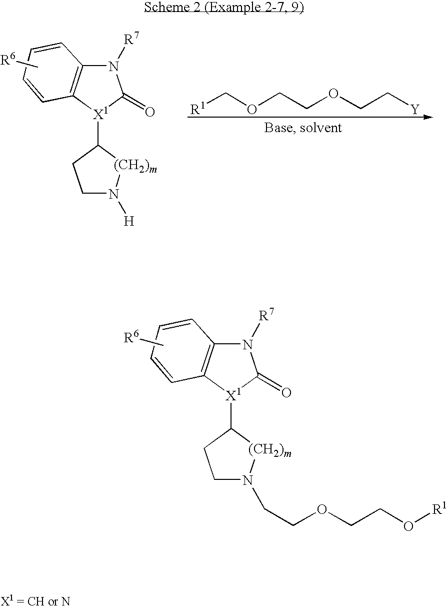

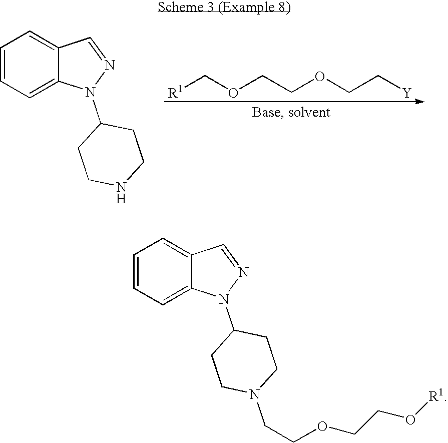

### Biological Evaluation

### Human M1, Rat M1, Human M3 and Human M5 Calcium Mobilization FLIPR™ Assay

The compound activity in the present invention (EC50 or IC₅₀) was measured using a 384 plate-based imaging assay that monitors drug induced intracellular Ca² release in whole cells. Activation of hM1 (human Muscarinic receptor subtype 1, gene bank access NM_{—}000738), rM1 (rat Muscarinic receptor subtype 1, gene bank access NM_{—}080773), hM3 (human Muscarinic receptor subtype 3, gene bank access NM_{—}000740NM_{—}000740) and hM5 (human Muscarinic receptor subtype 5, gene bank access NM_{—}0121258), receptors expressed in CHO cells (chinese hamster ovary cells, ATCC) was quantified in a Molecular Devices FLIPR II™ instrument as an increase in fluorescent signal. Inhibition of hM3 and hM5 by compounds was determined by the decrease in fluorescent signal in response to 20 nM carbachol activation.

CHO cells were plated in 384-black polylysine coated plate (Costar) at 8000 cells/well/50 μl for 24 hours or 4000 cells/well for 48 hours in a humidified incubator (5% CO₂ and 37° C.) in DMEM/F12 medium without selection agent. Prior to the experiment the cell culture medium was removed from the plates by inversion. A loading solution of 30 μl of Hank's balanced salt solution, 10 mM Hepes and 2.5 mM Probenicid at Ph 7.4 (Cat no. 311-520-VL, Wisent) with 2 μM calcium indicator dye (FLUO-3AM, Molecular Probes F14202) was added to each well. Plates were incubated at 37° C. for 60 minutes prior to start the experiment. The incubation was terminated by washing the cells four times in assay buffer, leaving a residual 25 μl buffer per well. Cell plates were then transferred to the FLIPR, ready for compound additions.

The day of experiment, carbachol and compounds were diluted in three-fold concentration range (10 points serial dilution) for addition by FLIPR instrument. For all calcium assays, a baseline reading was taken for 30 seconds followed by the addition of 12.5 μl (25 μl for hM1 and rM1) of compounds, resulting in a total well volume of 37.5 μl (50 μl for hM1 and rM1). Data were collected every 1.6 seconds for 300 seconds. For hM3 and hM5 an additional 12.5 μl of carbachol (20 nM final) was added at 300 seconds. After this addition of carbachol (producing a final volume of 50 μl), the FLIPR continued to collect data every 2 seconds for 240 seconds. The fluorescence emission was read using filter 1 (emission 520-545 nm) by the FLIPR on board CCD camera.

Calcium mobilization output data were calculated as the maximal relative fluorescence unit (RFU) minus the minal value for both compound and agonist reading frame (except for hM1 and rM1 using only the maximal RFU). Data were analyzed using sigmoidal fits of a non-linear curve-fitting program (XLfit version 5.0.6 from ID Business Solutions Limited, Guildford, UK). All EC50 and 1050 values are reported as arithmetic means±standard error of mean of ‘n’ independent experiments. Using the above-mentioned assays, the IC50 and EC50 towards human hM1, ratM1, hM3 and hM5 receptors for most compounds is measured to be in the range 1->30000 nM. The Eₘₐₓ (maximal effect, agonism or antagonist inhibition) towards human hM1, ratM1, hM3 and hM5 receptors for most compounds is measured to be in the range of 0-110%.

### hM2 Receptor GTPγS Binding

Membranes produced from Chinese hamster ovary cells (CHO) expressing the cloned human M2 receptor (human Muscarinic receptor subtype 2, gene bank access NM_{—}000739), were obtained from Perkin-Elmer (RBHM2M). The membranes were thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention were evaluated from 10-point dose-response curves (three fold concentration range) done in 60 μl in 384-well non-specific binding surface plate (Corning). Ten microliters from the dose-response curves plate (5× concentration) were transferred to another 384 well plate containing the following: 10 μg of hM2 membranes, 500 μg of Flashblue beads (Perkin-Elmer) and GDP in a 25 μl volume. An additional 15 μl containing 3.3× (55000 dpm) of GTPγ³⁵S (0.4 nM final) were added to the wells resulting in a total well volume of 50 μl. Basal and maximal stimulated [³⁵S]GTPγS binding were determined in absence and presence of 30 μM of acetylcholine agonist. The membranes/beads mix were pre-incubated for 15 minutes at room temperature with 25 μM GDP prior to distribution in plates (12.5 μM final). The reversal of acetylcholine-induced stimulation (2 μM final) of [³⁵S]GTPγS binding was used to assay the antagonist properties (IC₅₀) of the compounds. The plates were incubated for 60 minutes at room temperature with shaking, then centrifuged at 2000 rpm for 5 minutes. The radioactivity (cpm) were counted in a Trilux (Perkin-Elmer).

Values of EC₅₀, IC₅₀ and Eₘₐₓ were obtained using sigmoidal fits of a non-linear curve-fitting program (XLfit version 5.0.6 from ID Business Solutions Limited, Guildford, UK) of percent stimulated [³⁵S]GTRγS binding vs log(molar ligand).

All EC50 and IC50 values are reported as arithmetic means t standard error of mean of ‘n’ independent experiments. Based on the above assays, the EC₅₀ towards human M2 receptors for most compounds of the invention is measured to be in the range of about between 200 and >30000 nM. The Eₘₐₓ (maximal effect, agonism or antagonist inhibition) towards human M2 receptors for most compounds of the invention were measured to be in the range of about 0-120%. The IC₅₀ was the concentration of the compound of the invention at which 50% inhibition of acetylcholine [³⁵S]GTPγS binding stimulation has been observed. The IC₅₀ towards human M2 receptors for most compounds of the invention was measured to be in the range of between 40 and >90000 nM.

### HM4 Receptor GTPγS Binding

Membranes produced from Chinese hamster ovary cells (CHO) expressing the cloned human M4 receptor (human Muscarinic receptor subtype 4, gene bank access NM_{—}000741), were obtained from Perkin-Elmer (RBHM4M). The membranes were thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention were evaluated from 10-point dose-response curves (three fold concentration range) done in 60 μl in 384-well non-specific binding surface plate (Corning). Ten microliters from the dose-response curves plate (5× concentration) were transferred to another 384 well plate containing the following: 10 μg of hM4 membranes, 500 μg of Flashblue beads (Perkin-Elmer) and GDP in a 25 μl volume. An additional 150 containing 3.3× (55000 dpm) of GTPγ³⁵S (0.4 nM final) were added to the wells resulting in a total well volume of 50 μl. Basal and maximal stimulated [³⁵S]GTPγS binding were determined in absence and presence of 30 μM of acetylcholine agonist. The membranes/beads mix were pre-incubated for 15 minutes at room temperature with 40 μM GDP prior to distribution in plates (20 μM final). The reversal of acetylcholine-induced stimulation (10 μM final) of [³⁵S]GTPγS binding was used to assay the antagonist properties (IC₅₀) of the compounds. The plates were incubated for 60 minutes at room temperature with shaking, then centrifuged at 2000 rpm for 5 minutes. The radioactivity (cpm) were counted in a Trilux (Perkin-Elmer).

Values of EC₅₀, IC₅₀ and Eₘₐₓ were obtained using sigmoidal fits of a non-linear curve-fitting program (XLfit version 5.0.6 from ID Business Solutions Limited, Guildford, UK) of percent stimulated [³⁵S]GTPγS binding vs log(molar ligand).

All EC50 and IC₅₀ values are reported as arithmetic means t standard error of mean of ‘n’ independent experiments. Based on the above assays, the EC₅₀ towards human M4 receptors for most compounds of the invention is measured to be in the range of between 300 and >30000 nM. The Eₘₐₓ (maximal effect, agonism or antagonist inhibition) towards human M4 receptors for most compounds of the invention were measured to be in the range of about 0-120%. The IC₅₀ was the concentration of the compound of the invention at which 50% inhibition of acetylcholine [³⁵S]GTPγS binding stimulation has been observed. The IC₅₀ towards human M4 receptors for most compounds of the invention was measured to be in the range of between 3000 and >30000 nM.

### EXAMPLES

The invention will further be described in more detail by the following Examples which describe methods whereby compounds of the present invention may be prepared, purified, analyzed and biologically tested, and which are not to be construed as limiting the invention.

### Example 1

### 1-(1-{[5-(methoxymethyl)-2-furyl]methyl}piperidin-4-yl)-1,3-dihydro-2H-benzimidazol-2-one

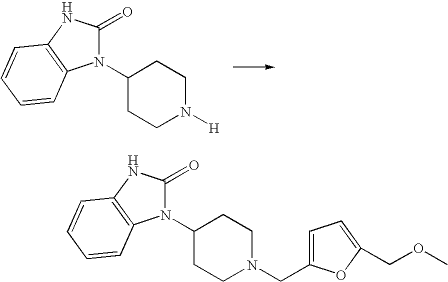

Acetic acid (0.6 mL) was added dropwide to a mixture of 1-piperidin-4-yl-1,3-dihydro-2H-benzimidazol-2-one (684 mg, 3.15 mmol), 5-(methoxymethyl)-2-furaldehyde (440 mg, 3.14 mmol), NaBH(OAc)₃ (680 mg, 3.22 mmol) in dichloromethane (20 mL). The mixture was stirred at room temperature for 48 h. Usual work and purification on prep-HPLC afforded the title compound, which was converted to its HCl salt (620 mg). MS (M+1): 342.08. 1H NMR (400 MHz, METHANOL-D4): δ ppm 2.07 (d, J=13.28 Hz, 2H), 2.69-2.92 (m, 2H), 3.19-3.32 (m, 3H), 3.35 (s, 3H), 3.66 (d, J=12.30 Hz, 2H), 4.42 (s, 2H), 4.46 (s, 2H), 4.50-4.62 (m, 1H), 6.49 (d, J=3.13 Hz, 1H), 6.74 (d, J=3.13 Hz, 1H), 7.01-7.11 (m, 3H), 7.29-7.37 (m, 1H).

### Example 2

### 1-{1-[2-(2-methoxyethoxy)ethyl]piperidin-4-yl}-1,3-dihydro-2H-benzimidazol-2-one

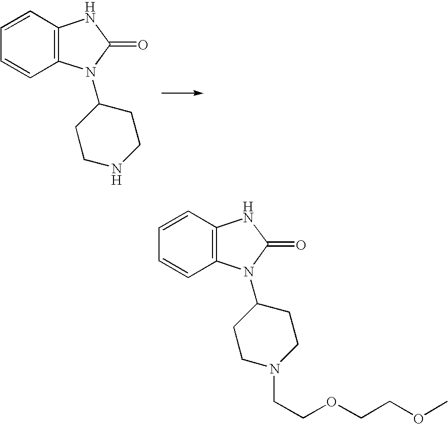

The mixture of 1-bromo-2-(2-methoxyethoxy)ethane (2.4 mmol), 1-piperidin-4-yl-1,3-dihydro-2H-benzimidazol-2-one (2.00 mmol), sodium carbonate (5 mmol) in acetonitrile (10 mL) was heated at reflux overnight under nitrogen. The reaction mixture was allowed to cool to room temperature, diluted with ether (20 mL). The solid was filtered off, and the solvent was concentrated. The residue was re-dissolved in ether (30 mL), and treated with 2N HCl in ether. The precipitate was collected and dried to afford the title compound. MS (M+1): 320.05. 1H NMR (400 MHz, DMSO-D6): δ ppm 1.82 (d, J=12.50 Hz, 2H), 2.80 (d, J=12.50 Hz, 2H), 3.04-3.31 (m, 4H), 3.37-3.71 (m, 8H), 3.82 (s, 2H), 4.51 (t, J=12.11 Hz, 1H), 6.96 (s, 3H), 7.57 (s, 1H), 10.39-11.20 (m, 1H), 10.93 (s, 1H).

### Example 3

### 1-{1-[2-(2-ethoxyethoxy)ethyl]piperidin-4-yl}-1,3-dihydro-2H-benzimidazol-2-one

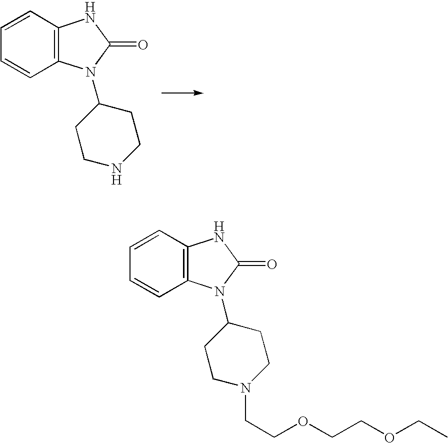

The mixture of 1-piperidin-4-yl-1,3-dihydro-2H-benzimidazol-2-one (100 mg, 0.46 mmol), potassium carbonate (250 mg, 1.81 mmol) and 1-bromo-2-(2-ethoxyethoxy)ethane (0.1 mL, 0.64 mmol) in acetonitrile (15 mL) was heated at 50° C. for 48 hours. The reaction mixture was concentrated under reduced pressure. The residue was dissolved diluted in dichloromethane, washed with water and dried. The crude product was purified by prep LCMS (acetonitrile/water) to provide the pure compound as its TFA salt (39%). 1H NMR (400 MHz, CHLOROFORM-D): δ ppm 1.16 (t, J=7.03 Hz, 3H), 1.95 (d, J=13.28 Hz, 2H), 2.83-3.09 (m, 4H), 3.26-3.39 (m, 2H), 3.49 (q, J=7.03 Hz, 2H), 3.52-3.59 (m, 2H), 3.58-3.68 (m, 2H), 3.78-4.02 (m, 4H), 4.52-4.75 (m, 1H), 6.84-7.15 (m, 3H), 7.41 (d, J=7.03 Hz, 1H), 10.16 (s, 1H). MS: 334.0 (M+1).

### Example 4

### 1-{1-[2-(2-ethoxyethoxy)ethyl]pyrrolidin-3-yl}-1,3-dihydro-2H-benzimidazol-2-one

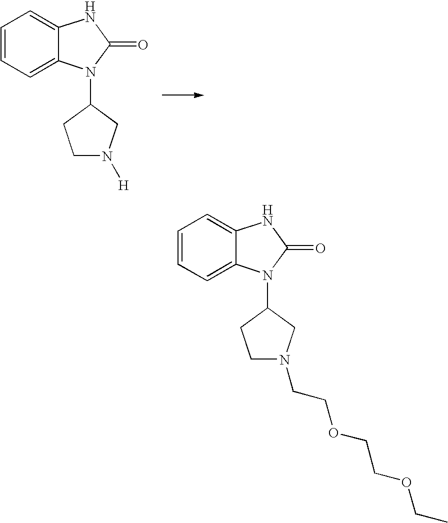

Following the procedure described in Example 3, the title compound (35 mg, 10%) was prepared from 1-pyrrolidin-3-yl-1,3-dihydro-2H-benzimidazol-2-one (0.25 g, 1.15 mmol) and 1-bromo-2-(2-ethoxyethoxy)ethane (0.21 mL, 1.35 mmol). MS: 320.3 (M+1). 1H NMR (400 MHz, CHLOROFORM-D): δ ppm 1.17 (t, J=7.03 Hz, 3H), 2.11-2.33 (m, 2H), 2.44-2.55 (m, 1H), 2.63-2.75 (m, 2H), 2.81-2.90 (m, 1H), 3.10-3.20 (m, 2H), 3.46-3.54 (m, 2H), 3.55-3.60 (m, 2H), 3.60-3.69 (m, 4H), 5.14-5.24 (m, 1H), 7.00-7.07 (m, 2H), 7.07-7.13 (m, 1H), 7.67-7.74 (m, 1H). MS: 320.3 (M+1).

### Example 5

### 5-chloro-1-{1-[2-(2-ethoxyethoxy)ethyl]piperidin-4-yl}-1,3-dihydro-2H-benzimidazol-2-one

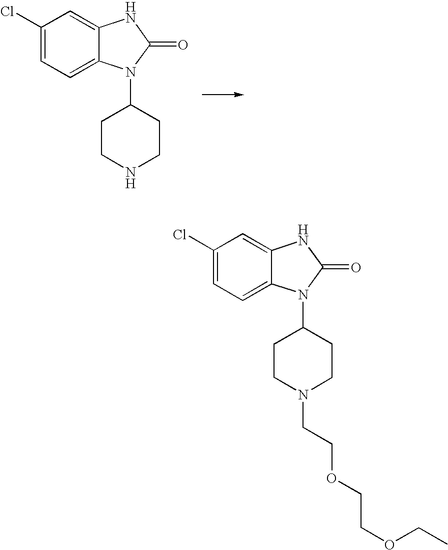

Following the similar procedure of Example 3, the title compound was prepared from 5-chloro-1-piperidin-4-yl-1,3-dihydro-2H-benzimidazol-2-one and 1-bromo-2-(2-ethoxyethoxy)ethane. HCl salt: 1H NMR (400 MHz, METHANOL-D4): δ ppm 1.11 (t, J=7.03 Hz, 3H), 1.99 (d, J=13.48 Hz, 2H), 2.77 (dq, J=13.38, 13.18, 3.81 Hz, 2H), 3.15-3.25 (m, 2H), 3.28-3.35 (m, 2H), 3.47 (q, J=7.03 Hz, 2H), 3.52-3.58 (m, 2H), 3.57-3.64 (m, 2H), 3.66-3.76 (m, 2H), 3.76-3.82 (m, 2H), 4.40-4.55 (m, 1H), 6.95-7.02 (m, 2H), 7.19-7.29 (m, 1H). MS: 368.3 (M+1).

### Example 6

### 3-(1-[2-(2-ethoxyethoxy)ethyl]piperidin-4-yl)-1,3-dihydro-2H-indol-2-one

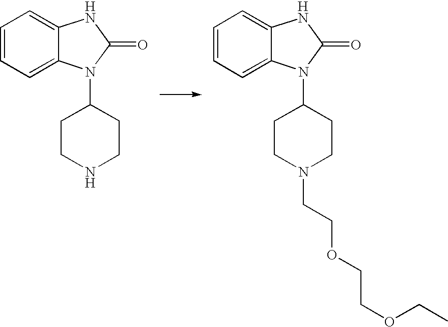

Following the similar procedure of Example 3, the title compound was prepared from 3-piperidin-4-yl-1,3-dihydro-2H-indol-2-one and 1-bromo-2-(2-ethoxyethoxy)ethane. MS (M+1): 333.3. 1H NMR (400 MHz, METHANOL-D4): δ ppm 1.14 (t, J=7.03 Hz, 3H) 1.45-1.62 (m, 2H) 1.64-1.88 (m, 2H) 2.04-2.26 (m, 3H) 2.59-2.67 (m, 2H) 3.07 (dd, J=24.51, 12.01 Hz, 2H) 3.26-3.44 (m, 1H) 3.49 (q, J=7.03 Hz, 2H) 3.53-3.56 (m, 5H) 3.59 (t, J=5.76 Hz, 2H) 6.86 (d, J=7.62 Hz, 1H) 6.94-7.03 (m, 1H) 7.18 (t, J=7.71 Hz, 1H) 7.28 (d, J=7.42 Hz, 1H)

### Example 7

### 1-{1-[2-(2-isopropoxyethoxy)ethyl]piperidin-4-yl}-1,3-dihydro-2H-benzimidazol-2-one

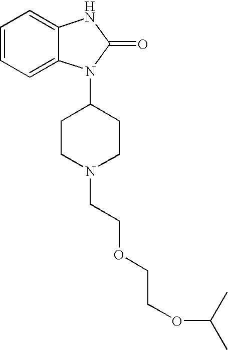

Step 1. The preparation of 2-[2-(2-chloroethoxy)ethoxy]propane

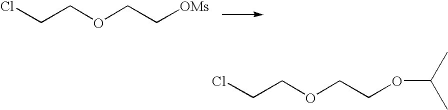

Dry isopropyl alcohol (5 mL) was placed in a dried 4-necked flask and metallic sodium (114 mg, 4.96 mmol) was added under a steam of nitrogen. The mixture was heated under reflux for 3 hours. Then, heating was stopped and 2-(2-chloroethoxy)ethyl methanesulfonate (1 g), which was prepared similar to known procedure reported in EP448078 A2 (1991), was added all at once and the mixture was refluxed over night. The mixture was then cooled to room temperature and 10% HCl solution was added and the compound extracted twice with ether. The organic layer was washed with an aq. saturated solution of NaHCO₃ then with a saturated aq. solution of NaCl and dried over Na₂SO₄ and condensed under reduced pressure. The residue was then used for the next step without any purification.

Step 2. The preparation of 1-{1-[2-(2-isopropoxyethoxy)ethyl]piperidin-4-yl}-1,3-dihydro-2H-benzimidazol-2-one

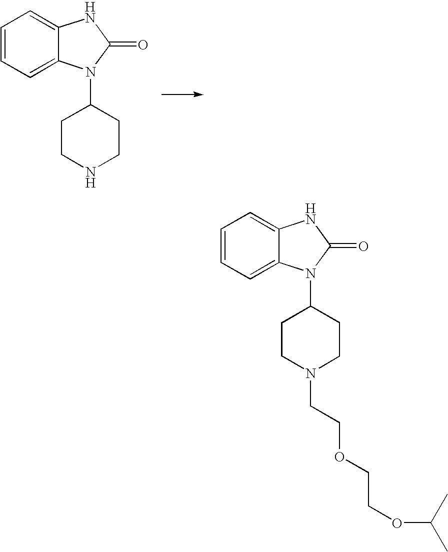

1-piperidin-4-yl-1,3-dihydro-2H-benzimidazol-2-one (260 mg, 1.20 mmol) was dissolved in ethanol (3 mL). To this solution was added NaI (36 mg, 0.24 mmol), Na2CO3 (166 mg, 1.57 mmol) and 2-[2-(2-chloroethoxy)ethoxy]propane. The mixture was heated at 80° C. for 48 hours. The mixture was then filtered and condensed under reduced pressure. The residue was diluted with dichloromethane and washed with water and an aq. sat. solution of NaCl and dried over Na2SO4. The solvent was then removed under reduced pressure and the residue was purified with prep LCMS to obtain 102 mg (18%) of the title compound as TFA salt as a white solid. MS: 348.2 (M+1). 1H NMR (400 MHz, METHANOL-D4): δ ppm 1.12 (d, J=6.05 Hz, 6H), 2.02 (d, J=13.09 Hz, 2H), 2.82 (qd, J=13.43, 13.30, 3.71 Hz, 2H), 3.16-3.28 (m, 2H), 3.33-3.41 (m, 2H), 3.51-3.68 (m, 5H), 3.72-3.95 (m, 4H), 4.42-4.63 (m, 1H), 6.97-7.10 (m, 3H), 7.20-7.36 (m, 1H). MS: 348.2 (M+1).

### Example 8

### 1-{1-[2-(2-ethoxyethoxy)ethyl]piperidin-4-yl}-1H-Indazole

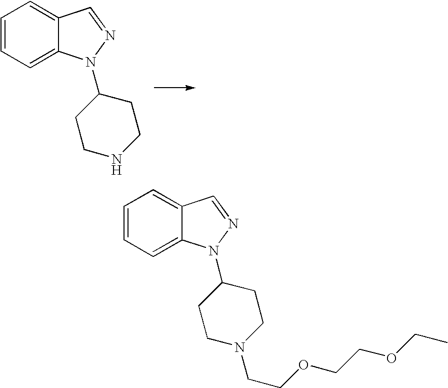

To a solution of 1-piperidin-4-yl-1H-indazole TFA salt (0.33 mmol) in DMF (10 ml) was added K₂CO₃ (137 mg, 0.99 mmol) followed by 1-bromo-2-(2-ethoxyethoxy)ethane (72 mg, 0.363 mmol). The mixture was heated under N₂ atmosphere at 75° C. for 5 hours, cooled down at room temperature and the solvent was evaporated in vacuo. The residue was dissolved in EtOAc (20 ml) and washed with saturated NaHCO₃ (10 ml), brine (10 ml) and dried over sodium sulfate to give the crude compound, which was purified by prep LC/MS—high pH (40-70% acetonitrile/water to give desired compound as free base. The pure compound was dissolved in CH₂Cl₂ (5 ml) under N₂ atmosphere and 1M solution of hydrochloride in Et₂O was added (5 eq.). The mixture was stirred at room temperature for 5 minutes and the solvent was evaporated in vacuo to give the desired compound as HCl salt (76 mg, 65.08%). MS (M+1): 318.3; 1H NMR (400 MHz, METHANOL-D4): δ ppm 1.21 (t, J=7.03 Hz, 3H), 2.28 (d, J=13.09 Hz, 2H), 2.57 (q, J=13.67 Hz, 2H), 3.36 (br t, J=12.89 Hz, 2H), 3.44 (br t, J=4.69 Hz, 2H), 3.57 (q, J=7.03 Hz, 2H), 3.61-3.67 (m, 2H), 3.68-3.73 (m, 2H), 3.83 (br s, 1H), 3.85-3.90 (m, 7H), 5.00 (tt, J=11.40, 7.74, 3.91, 3.71 Hz, 1H), 7.14-7.21 (m, 1H), 7.43 (t, J=7.32 Hz, 1H), 7.67 (d, J=8.59 Hz, 1H), 7.75 (d, J=7.81 Hz, 1H), 8.05 (s, 1H).

### Example 9

### 1-{1-[2-(2-ethoxyethoxy)ethyl]piperidin-4-yl}-3-methyl-1,3-dihydro-2H-benzimidazol-2-one

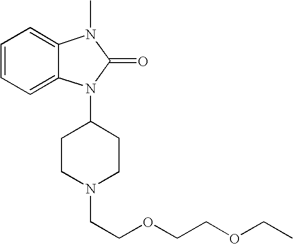

Step A. tert-butyl 4-(2-oxo-2,3-dihydro-1H-benzimidazol-1-yl)piperidine-1-carboxylate

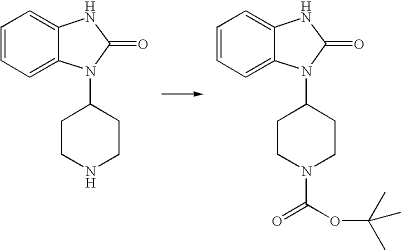

To a solution of 1-piperidin-4-yl-1,3-dihydro-2H-benzimidazol-2-one (1.5 g, 6.91 mmol) in acetone was added Boc₂O (1.5 g, 6.91 mmol). The mixture stirred over night and then condensed under reduced pressure to provide a white solid (2.2 g). The crude was used without any purification for the next step. MS: 318.1 (M+1).

Step B. 1-methyl-3-piperidin-4-yl-1,3-dihydro-2H-benzimidazol-2-one

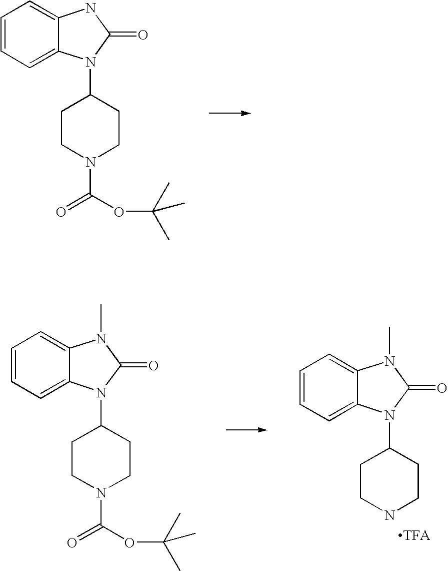

To a solution of tart-butyl 4-(2-oxo-2,3-dihydro-1H-benzimidazol-1-yl)piperidine-1-carboxylate (500 mg, 1.57 mmol) in DMF (10 mL) was added NaH (126 mg, 3.15 mmol) and the mixture was stirred for one hour. Methyl iodide (0.128 uL, 2.05 mmol) was then added drop-by-drop and stirred for 3 hours. Quenched with ice-water and then condensed under reduced pressure. Diluted in dichloromethane and washed with water, and with a saturated solution of NaCl then dried and condensed under reduced pressure. MS: 332.1 (M+1).

To the above crude was added dichloromethane (5 mL) and TFA (2 mL) and stirred at room temperature for one hour. The mixture was condensed under reduced pressure to provide a yellow-pale solid, which used for next step without any purification (0.8 g). MS: 231.9 (M+1).

Step C. The preparation of 1-(1-[2-(2-ethoxyethoxy)ethyl]piperidin-4-yl)-3-methyl-1,3-dihydro-2H-benzimidazol-2-one

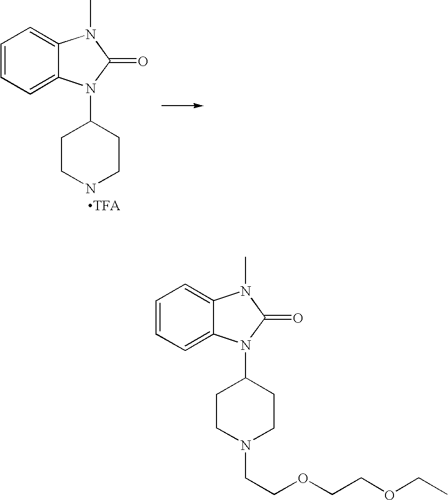

Following the procedure described in Example 3, the title compound was prepared from 1-methyl-3-piperidin-4-yl-1,3-dihydro-2H-benzimidazol-2-one and 1-bromo-2-(2-ethoxyethoxy)ethane. 1H NMR (400 MHz, CHLOROFORM-D): δ ppm 1.20 (t, J=7.03 Hz, 3H), 1.77 (d, J=13.09 Hz, 2H), 2.16-2.34 (m, 1H), 2.38-2.56 (m, 1H), 2.56-2.62 (m, 1H), 2.60 (s, 3H), 2.61-2.77 (m, 1H), 3.03-3.18 (m, 1H), 3.40 (s, 3H), 3.53 (q, J=6.90 Hz, 2H), 3.56-3.61 (m, 2H), 3.60-3.70 (m, 4H), 4.34-4.44 (m, 1H), 6.92-6.98 (m, 1H), 7.01-7.11 (m, 2H), 7.28-7.35 (m, 1H). MS: 348.3 (M+1).

1. A compound of formula I, a pharmaceutically acceptable salt thereof, diastereomer, enantiomer, or mixture thereof: 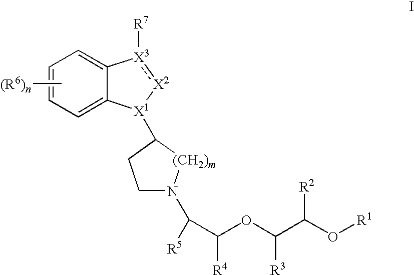
wherein
R¹ is selected from hydrogen, C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂; R², R³ and R⁴ are independently selected from hydrogen, C₁₋₆alkyl, C₂₋₆alkenyl, and C₁₋₆alkoxy, wherein said C₁₋₆alkyl, C₂₋₆alkenyl, and C₁₋₆alkoxy are optionally substituted by one or more groups selected from hydroxy, amino, halogen, C₁₋₆alkoxy and —CN; or R³ and R⁴ together with the atoms connected thereto form a 5 to 7 membered C₂₋₆heterocycloalkyl or C₂₋₆heteroaryl; or R¹ and R² together with the atoms connected thereto form a 5 to 7 membered C₂₋₆heterocycloalkyl or C₂₋₆heteroaryl, wherein said C₂₋₆heterocycloalkyl or C₂₋₆heteroaryl is optionally substituted with one or more group selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₚOR, R, —CO₂R; —SO₂R; —SO₂NR₂, halogen, —NO₂, —NR₂, —(CH₂)ₚNR₂, and —C(═O)—NR₂;
R⁵ is selected from hydrogen, halogen, hydroxy, C₁₋₆alkyl, C₂₋₆alkenyl, and C₁₋₆alkoxy, wherein said C₁₋₆alkyl, C₂₋₆alkenyl, and C₁₋₆alkoxy are optionally substituted by one or more groups selected from hydroxy, amino, halogen, C₁₋₆alkoxy and —CN;
R⁶ is independently selected from hydrogen, halogen, C₁₋₆alkyl, C₂₋₆alkenyl, —CN, —C(═O)—OR, —C(═O)—NR₂, hydroxy, and C₁₋₆alkoxy;
R⁷ is selected from hydrogen, C₁₋₆alkyl, C₂₋₆alkenyl, halogen and C₁₋₆alkoxy;
n is 1, 2, 3 or 4;
m is 1, 2 or 3;
p is 1, 2, 3 or 4;
each R is independently hydrogen, C₁₋₆alkyl, C₂₋₆alkenyl or halogenated C₁₋₆alkyl; and
X¹, X² and X³ is independently selected from C(═O), NH, N, CH₂, and CH, wherein at least one of X¹, X² and X³ is selected from NH and N; wherein at most one of X¹, X² and X³ is C(═O); and wherein X¹ is not C(═O).

2. A compound as claimed in claim 1, wherein
R¹ and R² of formula I together with the atoms connected thereto form a 5 to 7 membered C₂₋₆heterocycloalkyl, wherein said C₂₋₆heterocycloalkyl is optionally substituted with one or more groups selected from C₁₋₆alkyl, C₁₋₆alkoxy, hydroxy, halogen, amine and amido.

3. A compound as claimed in claim 1, wherein
R³ and R⁴ of formula I together with the atoms connected thereto form a 5 to 7 membered C₂₋₆heteroaryl, wherein said C₂₋₆heteroaryl is optionally substituted with one or more groups selected from C₁₋₆alkyl, C₁₋₆alkoxy, hydroxy, halogen, amine and amido.

4. A compound as claimed in claim 1, wherein
R¹, R², R³ and R⁴ are independently selected from hydrogen, C₁₋₆alkyl, C₂₋₆alkenyl, C₁₋₆alkyl-carbonyl, C₁₋₆alkylaminocarbonyl, and C₁₋₆alkoxy, wherein said C₁₋₆alkyl, C₂₋₆alkenyl, C₁₋₆alkyl-carbonyl, C₁₋₆alkylaminocarbonyl, and C₁₋₆alkoxy are optionally substituted by one or more groups selected from amino, halogen, hydroxy, C₁₋₆alkoxy and —CN.

5. A compound as claimed in claim 1, wherein
R¹, R², R³ and R⁴ are independently selected from hydrogen, and C₁₋₃alkyl.

6. A compound as claimed in claim 1, wherein R⁵ is selected from hydrogen and C₁₋₆alkyl optionally substituted by one or more groups selected from hydroxy, amino, halogen, C₁₋₆alkoxy and —CN.

7. A compound as claimed in claim 1, wherein R⁵ is hydrogen.

8. A compound as claimed in claim 1, wherein R⁶ is selected from hydrogen, halogen, methyl, ethyl, —CN, —C(═O)—NH₂, —CO₂CH₃, —CO₂H, hydroxy and methoxy.

9. A compound as claimed in claim 1, wherein m is 2.

10. A compound as claimed in claim 1, wherein p is 1.

11. A compound as claimed in claim 1, wherein n is 1.

12. A compound as claimed in claim 1, wherein m is 1.

13. A compound as claimed in claim 1, wherein p is 2.

14. A compound as claimed in claim 1, wherein X¹ is selected from N and CH.

15. A compound as claimed in claim 1, wherein X² is selected from N and C(═O).

16. A compound as claimed in claim 1, wherein X³ is selected from N and CH.

17. A compound as claimed in claim 1, wherein R⁷ is selected from hydrogen, C₁₋₆alkyl and halogen.

18. A compound as claimed in claim 1, wherein R⁷ is hydrogen.

19. A compound selected from
1-(1-{[5-(methoxymethyl)-2-furyl]methyl}piperidin-4-yl)-1,3-dihydro-2H-benzimidazol-2-one;
1-{1-[2-(2-methoxyethoxy)ethyl]piperidin-4-yl}-1,3-dihydro-2H-benzimidazol-2-one;
1-{1-[2-(2-ethoxyethoxy)ethyl]piperidin-4-yl}-1,3-dihydro-2H-benzimidazol-2-one;
1-{1-[2-(2-ethoxyethoxy)ethyl]pyrrolidin-3-yl}-1,3-dihydro-2H-benzimidazol-2-one;
5-chloro-1-{1-[2-(2-ethoxyethoxy)ethyl]piperidin-4-yl}-1,3-dihydro-2H-benzimidazol-2-one;
3-{1-[2-(2-ethoxyethoxy)ethyl]piperidin-4-yl}-1,3-dihydro-2H-indol-2-one;
1-{1-[2-(2-isopropoxyethoxy)ethyl]piperidin-4-yl}-1,3-dihydro-2H-benzimidazol-2-one;
1-{1-[2-(2-ethoxyethoxy)ethyl]piperidin-4-yl}-1H-indazole;
1-{1-[2-(2-ethoxyethoxy)ethyl]piperidin-4-yl}-3-methyl-1,3-dihydro-2H-benzimidazol-2-one;
and pharmaceutically acceptable salts thereof.

20. A compound of formula II, a pharmaceutically acceptable salt thereof, diastereomer, enantiomer, or mixture thereof: 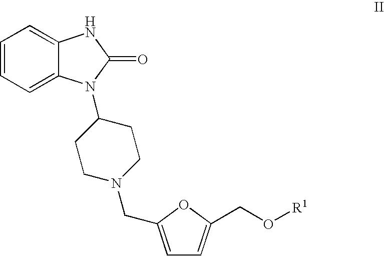
wherein
R¹ is selected from hydrogen, C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂, wherein p is 1, 2, 3 or 4; and each R is independently hydrogen, C₁₋₆alkyl, C₂₋₆alkenyl or halogenated C₁₋₆alkyl.

21. A compound as claimed in claim 20, wherein
R¹ is selected from hydrogen, C₁₋₆alkyl-carbonyl, C₁₋₆alkylaminocarbonyl, C₁₋₆alkyl and C₂₋₆alkenyl, wherein said C₁₋₆alkyl-carbonyl, C₁₋₆alkylaminocarbonyl, C₁₋₆alkyl and C₂₋₆alkenyl are optionally substituted by one or more groups selected from fluoro, chloro, bromo, methoxy, hydroxy and —CN.

22. A compound as claimed in claim 20, wherein said R¹ is selected from hydrogen and C₁₋₃alkyl.

23. A compound of formula III, a pharmaceutically acceptable salt thereof, diastereomer, enantiomer, or mixture thereof: 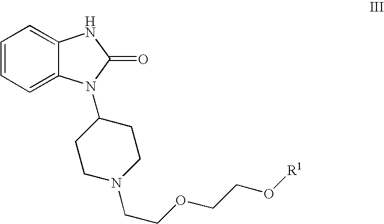
wherein
R¹ is selected from hydrogen, C₁₋₆alkyl, C₂₋₆alkenyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂, wherein p is 1, 2, 3 or 4; and each R is independently hydrogen, C₁₋₆alkyl, C₂₋₆alkenyl or halogenated C₁₋₆alkyl.

24. A compound as claimed in claim 23, wherein
R¹ is selected from hydrogen, C₁₋₆alkyl and C₂₋₆alkenyl, wherein said C₁₋₆alkyl and C₂₋₆alkenyl are optionally substituted by one or more groups selected from fluoro, chloro, bromo, methoxy, hydroxy and —CN.

25. A compound as claimed in claim 23, wherein
R¹ of formula III is selected from hydrogen and C₁₋₃alkyl.

26. A compound of formula IV, a pharmaceutically acceptable salt thereof, diastereomer, enantiomer, or mixture thereof: 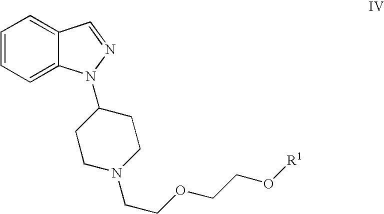
wherein
R¹ is selected from hydrogen, C₁₋₆alkyl, C₂₋₆alkenyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂, wherein p is 1, 2, 3 or 4; and each R is independently hydrogen, C₁₋₆alkyl, C₂₋₆alkenyl or halogenated C₁₋₆alkyl.

27. A compound as claimed in claim 26, wherein
R¹ is selected from hydrogen, C₁₋₆alkyl and C₂₋₆alkenyl, wherein said C₁₋₆alkyl and C₂₋₆alkenyl are optionally substituted by one or more groups selected from fluoro, chloro, bromo, methoxy, hydroxy and —CN.

28. A compound as claimed in claim 26, wherein
R¹ is selected from hydrogen and C₁₋₃alkyl.

29-32. (canceled)

33. A pharmaceutical composition comprising a compound according to claim 1 and a pharmaceutically acceptable carrier.

34. A method for the therapy of pain in a warm-blooded animal, comprising the step of administering to said animal in need of such therapy a therapeutically effective amount of a compound according to claim 1.

35. A method for the therapy of Alzheimer's disease in a warm-blooded animal, comprising the step of administering to said animal in need of such therapy a therapeutically effective amount of a compound according to claim 1.

36. A method for the therapy of schizophrenia in a warm-blooded animal, comprising the step of administering to said animal in need of such therapy a therapeutically effective amount of a compound according to claim 1.

37. A process for preparing a compound of Formula I, comprising: 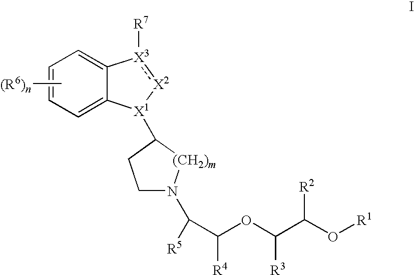
reacting a compound of Formula V with a compound of formula VI, 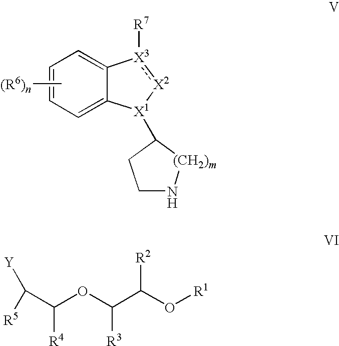
wherein Y is a halogen;
R¹ is selected from hydrogen, C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂; R², R³ and R⁴ are independently selected from hydrogen, C₁₋₆alkyl, C₂₋₆alkenyl, and C₁₋₆alkoxy, wherein said C₁₋₆alkyl, C₂₋₆alkenyl, and C₁₋₆alkoxy are optionally substituted by one or more groups selected from hydroxy, amino, halogen, C₁₋₆alkoxy and —CN; or R³ and R⁴ together with the atoms connected thereto form a 5 to 7 membered C₂₋₆heterocycloalkyl or C₂₋₆heteroaryl; or
R¹ and R² together with the atoms connected thereto form a 5 to 7 membered C₂₋₆heterocycloalkyl or C₂₋₆heteroaryl, wherein said C₂₋₆heterocycloalkyl or C₂₋₆heteroaryl is optionally substituted with one or more group selected from C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, —CN, —SR, —OR, —(CH₂)ₚOR, R, —CO₂R; —SO₂R; —SO₂NR₂, halogen, —NO₂, —NR₂, —(CH₂)ₚNR₂, and —C(═O)—NR₂;
R⁵ is selected from hydrogen, halogen, hydroxy, C₁₋₆alkyl, C₂₋₆alkenyl, and C₁₋₆alkoxy, wherein said C₁₋₆alkyl, C₂₋₆alkenyl, and C₁₋₆alkoxy are optionally substituted by one or more groups selected from hydroxy, amino, halogen, C₁₋₆alkoxy and —CN;
R⁶ is independently selected from hydrogen, halogen, C₁₋₆alkyl, C₂₋₆alkenyl, —CN, —C(═O)—OR, —C(═O)—NR₂, hydroxy, and C₁₋₆alkoxy;
R⁷ is selected from hydrogen, C₁₋₆alkyl, C₂₋₆alkenyl, halogen and C₁₋₆alkoxy;
n is 1, 2, 3 or 4;
m is 1, 2 or 3;
p is 1, 2, 3 or 4;
each R is independently hydrogen, C₁₋₆alkyl, C₂₋₆alkenyl or halogenated C₁₋₆alkyl; and
X¹, X² and X³ is independently selected from C(═O), NH, N, CH₂, and CH, wherein at least one of X¹, X² and X³ is selected from NH and N; wherein at most one of X¹, X² and X³ is C(═O); and wherein X¹ is not C(═O).

38. A process for preparing a compound of Formula II, comprising: 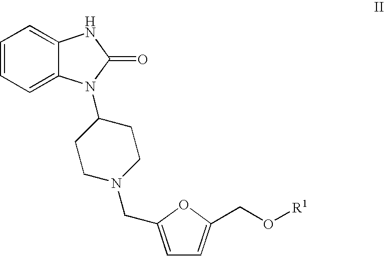
reacting 1-piperidin-4-yl-1,3-dihydro-2H-benzimidazol-2-one with a compound of formula VII, 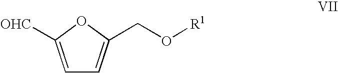
wherein R¹ is selected from hydrogen, C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂ and each R is independently hydrogen, C₁₋₆alkyl, C₂₋₆alkenyl or halogenated C₁₋₆alkyl.
